SEQUENCE LISTING (21 sequences)

SEQ ID NO: 1 - DNA, 774 bp, Staphylococcus aureus
atgaaaaaaacagcatttacattacttttattcattgccctaacgttgacaacaagtccacttgtaaatggtagcgagaaaagcgaagaaataaatgaaaaagatttgcgaaaaaagtctgaattgcagggaacagctttaggcaatcttaaacaaatctattattacaatgaaaaagctaaaactgaaaataaagagagtcacgatcaatttttacagcatactatattgtttaaaggcttttttacagatcattcgtggtataacgatttattagtagattttgattcaaaggatattgttgataaatataaagggaaaaaagtagacttgtatggtgcttattatggttatcaatgtgcgggtggtacaccaaacaaaacagcttgtatgtatggtggtgtaacgttacatgataataatcgattgaccgaagagaaaaaagtgccgatcaatttatggctagacggtaaacaaaatacagtacctttggaaacggttaaaacgaataagaaaaatgtaactgttcaggagttggatcttcaagcaagacgttatttacaggaaaaatataatttatataactctgatgtttttgatgggaaggttcagaggggattaatcgtgtttcatacttctacagaaccttcggttaattacgatttatttggtgctcaaggacagtattcaaatacactattaagaatatatagagataataaaacgattaactctgaaaacatgcatattgatatatatttatatacaagttaa

SEQ ID NO: 2 - protein, 257 aa, Staphylococcus aureus
MKKTAFTLLLFIALTLTTSPLVNGSEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYTS

SEQ ID NO: 3 - DNA, 1828 bp, Medicago sativa
tcgacctgcaggtcaacggatcaaatgattcaatatttggcttgatgaaattagagaaaatgaaaaattggatttctaagtttgattgttattttgagatagaaaaggaaaaatctctaatctcttacgcaagacctgcctcaaccacttgataaactcttttgtctacgtattgaaaacaaaagaggcaaataaacatctagccaaatgaaacaccaataatgctttaaacaaaatggaataattgcatcatcaattaatctttataagtgagaattttccctcctataataatgcgctaggtatcaattttcaactctgaaatataaagcttcaagcgtgtgttatcaaaaatcaagcacagtaaaatcataagcagaatcattggtgatgctaatagttgatgtggaatcgaacaatgttcatattctgataccttgttggtgacaagtcaagacccttatagacttgaattttgtctgagttgatgatttcagaaggggaatctagtatctaagtagatggtaaatttattttttccaattccagttgcttccattatgaacaaaccttattcttttaggctaatattgaggaacaaaagccacggaatattttttttatgtataacctaagaaaaagacaataataaaaataattaaaaactacaacagatgattttggacttgaatcgaattggattaatcttatacatgttgtcgataaggatactagttatatgaagaagagaatcaattgaaactttatttgtgctatatataatgatttatgatatatggagagagggatggcagaatatgcaagtttggaatcaattctggacattcatggagggcgggtttatcatcgtgggtgtggtaggggtggctgaggttctagggctacccagtacttttatgatgttgttgtagtattttgaacaaatttgttttttaattttatgtttgaatattgggtttgtaacatatggataatttgtttttaattttatgttcgaaaattgtgttgtttttgatcattttcattacaatatttatttatttattcacgaatgcatgtttatatcaacaaattatataatctgtatgtatcatagtgaaaacaaactctgtttttcttttgatacctttcagattatataatttgaaatgtcataaaacagtttagattatataatctgaaatatgcgttttttacaccacattttgcattttgtgaggtgtttgacacttttcggattatataagtcaaattattttaagaagtttcggattatatatctgaaacatatgtttaactgacacatacacaaacatctctagggtgatttgtcttccaatagtttttatactgtttggattatataatccgaatcaaggttaagaaaaaattagggcgctcgaaaaccaaatagggtgggaaaagtaatgaccaatattgattaccctataaggagccaaagcctgaaaaaagtaccatacatgattgatatttgtggaggcattaatagtcacaaaactacacgtggcaattttatattggtggctaatgataaggctagcacaaaaatttccattcctgtgtggttgatatggcagcaaagtttatcatattcacaaccaacaaaatggtattatgaagcattaccacaatttataagaccataatattggaaataggaaaataaaaacattatatatagcaagtttgagtataagctttgcaattcaagcagaagtacatcttactttactagtgaactaagtaagggagaaaaaaaatggcttcctctatgatgtcctcttca

SEQ ID NO: 4 - DNA, 240 bp, Medicago sativa
atggcttcctctatgctctcttccgctactatggttgcctctccggctcaggccactatggtcgctcctttcaacggacttaagtcctccgctgccttcccagccacccgcaaggctaacaacgacattacttccatcacaagcaacggcggaagagttaactgcatgcaggtgtggcctccgattggaaagaagaagtttgagactctctcttaccttcctgaccttaccgattccgaa

SEQ ID NO: 5 - protein, 80 aa, Medicago sativa
MASSMLSSATMVASPAQATMVAPFNGLKSSAAFPATRKANNDITSITSNGGRVNCMQVWPPIGKKKFETLSYLPDLTDSE

SEQ ID NO: 6 - DNA, 858 bp, Robinia pseudoacacia
atgacttcctacaacttcaaaacccaaacctccttccctcttctcctatccatatccttttttttcctcttgttactcaacaaggtgaattcaactggatctctctccttttctttccccaagttcgcgcctaaccaaccatatctgatcttccaacgtgatgcccttgtgacatcaacaggggtgttacaactcaccaacgtagttaacggggtaccatccggtaaatctcttggtagagctctatatgctgcccctttccaaatctgggatagcaccacaggcaacgtggctagctttgtcacttccttctcctttatcattcaagcacctaacccaaccacaacggcagatggtcttgccttctttcttgcaccagttgatactcagcccttagatgttggaggaatgctcggaattttcaaagacggatatttcaataaatccaaccaaattgttgcagttgaattcgataccttttcaaatattcactttgatccaaaaggtagacatatgggaatcaatgtcaactccatcgtgtccataaaaaccgtgccatggaattggacaaatggcgaagtagccaatgttttcataagctatgaagcttccaccaaatccttaactgcctctttggtttatccttcacttgaaacaagttttatcgttcatgctattgtggatgtgaaggatgttcttcccgagtgggtaagatttggtttctcagctaccacaggaatagataaaggctacgttcaaacaaatgatgttctctcctggtctttcgagtcaaacttgccaggtggtaacagtgttgcttcggtgaagaacgcgggtctttcaacctatgctgcatga

SEQ ID NO: 7 - protein, 285 aa, Robinia pseudoacacia
MTSYNFKTQTSFPLLLSISFFFLLLLNKVNSTGSLSFSFPKFAPNQPYLIFQRDALVTSTGVLQLTNVVNGVPSGKSLGRALYAAPFQIWDSTTGNVASFVTSFSFIIQAPNPTTTADGLAFFLAPVDTQPLDVGGMLGIFKDGYFNKSNQIVAVEFDTFSNIHFDPKGRHMGINVNSIVSIKTVPWNWTNGEVANVFISYEASTKSLTASLVYPSLETSFIVHAIVDVKDVLPEWVRFGFSATTGIDKGYVQTNDVLSWSFESNLPGGNSVASVKNAGLSTYAA

SEQ ID NO: 8 - DNA, 861 bp, Robinia pseudoacacia
atggcttcctacaagttcaaaacccaaaactccttccttcttctcctatccatatcctttttcttcctcttgttactcaacaaggtgaattcgactggatccctctccttttctttccccaagttcaagcatagccaaccagatctgatcttccaaagtgatgcccttgtgacatcaaaaggggtgttacaactcaccacggtaaatgatggaagaccagtctatgactctattggtcgagttctatatgctgcccctttccaaatttgggatagcaccactggcaacgtggctagctttgtcacttccttctcctttatcatcaaagcacctaacgaaggcaaaacggcagatggtcttgtcttctttcttgcaccagttggtagtactcagcccctaaaaggaggaggactcctcggacttttcaaagatgaatcttacaataaatccaaccaaattgttgcagttgaatttgacacatttcggaatgttgcatgggatccaaatggaatacatatgggaatcgatgtcaactctattcaatccgtaagaactgtgcgatgggattgggcgaatggcgaagtagccaatgttttcataagctatgaagcttccaccaaatccttaactgcctctttggtttatccttcacttgaaaaaagttttatcttgagtgctattgtggatttgaagaaagttcttccggagtgggtaagagttggtttcacagctaccacaggactatctgaagactacgttcaaacaaatgatgttctctcctggtctttcgagtcaaacttgccaggtggtaacagtgttgcttcggtgaagaacgcgggtctttcaacctatgctgcatga

SEQ ID NO: 9 - protein, 286 aa, Robinia pseudoacacia
MASYKFKTQNSFLLLLSISFFFLLLLNKVNSTGSLSFSFPKFKHSQPDLIFQSDALVTSKGVLQLTTVNDGRPVYDSIGRVLYAAPFQIWDSTTGNVASFVTSFSFIIKAPNEGKTADGLVFFLAPVGSTQPLKGGGLLGLFKDESYNKSNQIVAVEFDTFRNVAWDPNGIHMGIDVNSIQSVRTVRWDWANGEVANVFISYEASTKSLTASLVYPSLEKSFILSAIVDLKKVLPEWVRVGFTATTGLSEDYVQTNDVLSWSFESNLPGGNSVASVKNAGLSTYAA

SEQ ID NO: 10 - DNA, 783 bp, Robinia pseudoacacia
atgctcataagtttctttgtcttgctagctagtgccagaaaggagaactctgatgaaggaatttccttcaacttcaccaacttcaccagaggtgatcaaggtgtaaccttactaggacaagccaacattatggcaaatgggatcttggccctcaccaaccatacaaaccctacttggaatacaggccgtgccttgtattctaaaccagttcctatttgggattcagccactggcaatgtcgccagctttgttacttccttctcttttgtcgtacaagagatcaaaggtgctataccagctgatggaattgttttcttccttgcaccagaagccaggattcccgacaattcagccggtgggcaactcggaattgttaatgccaacaaagcttacaatccatttgttggtgtagaatttgatacttactccaataattgggatcctaaatctgcacatattggaatcgatgccagctctttaatttcattaaggactgtgaaatggaacaaggttagtgggtcattggtcaaagttagtatcatctatgactctctatctaagacgttgagtgttgttgtgactcacgagaatggtcaaatttctaccatcgctcaagtcgtggatttgaaagctgtgctgggagagaaggtcagggttggttttactgcagccaccacaacaggccgggaattatacgacattcatgcatggtctttcacttcaactttggtgacagctacaagcagcacctcgaagaacatgaatattgcaagctatgcatga

SEQ ID NO: 11 - protein, 260 aa, Robinia pseudoacacia
MLISFFVLLASARKENSDEGISFNFTNFTRGDQGVTLLGQANIMANGILALTNHTNPTWNTGRALYSKPVPIWDSATGNVASFVTSFSFVVQEIKGAIPADGIVFFLAPEARIPDNSAGGQLGIVNANKAYNPFVGVEFDTYSNNWDPKSAHIGIDASSLISLRTVKWNKVSGSLVKVSIIYDSLSKTLSVVVTHENGQISTIAQVVDLKAVLGEKVRVGFTAATTTGRELYDIHAWSFTSTLVTATSSTSKNMNIASYA

SEQ ID NO: 12 - DNA, 18 bp, Artificial Sequence
  Primer
atgacttcctacaacttc

SEQ ID NO: 13 - DNA, 18 bp, Artificial Sequence
  Primer
tcatgcagcataggttga

SEQ ID NO: 14 - DNA, 18 bp, Artificial Sequence
  Primer
atggcttcctacaagttc

SEQ ID NO: 15 - DNA, 18 bp, Artificial Sequence
  Primer
tcatgcagcataggttga

SEQ ID NO: 16 - DNA, 19 bp, Artificial Sequence
  Primer
atgctcataagtttctttg

SEQ ID NO: 17 - DNA, 18 bp, Artificial Sequence
  Primer
tcatgcatagcttgcaat

SEQ ID NO: 18 - DNA, 20 bp, Artificial Sequence
  Primer
caccaacttcaccagaggtg

SEQ ID NO: 19 - DNA, 22 bp, Artificial Sequence
  Primer
cacggcctgtattccaagtagg

SEQ ID NO: 20 - DNA, 21 bp, Artificial Sequence
  Primer
gcatctgcctcgacatcttga

SEQ ID NO: 21 - DNA, 21 bp, Artificial Sequence
  Primer
gacagcagcaccttggaaatg

(19) United States
(12) Patent Application Publication
(10) Pub. No.: US 2014/0283220 A1
(43) Pub. Date: Sep. 18, 2014

(54) Genetically Modified Plants that are Herbivore-Resistant

(71) Applicants: Yi Li, Mansfield, CT (US); Lisa Hollister, New Richmond, OH (US)

(72) Inventors: Yi Li, Mansfield, CT (US); Lisa Hollister, New Richmond, OH (US)

(21) Appl. No.: 14/212,834

(22) Filed: Mar. 14, 2014

Related U.S. Application Data
(60) Provisional application No. 61/862,148, filed on Aug. 5, 2013.
(60) Provisional application No. 61/787,514, filed on Mar. 15, 2013.

(51) Int. Cl. C12N 15/82; A01H 5/08; A01H 5/10

(52) U.S. Cl. CPC C12N 15/8271; A01H 5/10; A01H 5/0831
USPC 800/298; 435/320.1

(57) ABSTRACT

An herbivore-resistant transgenic plant. The plant may be transformed with a gene construct including at least one gene chosen from SEA, LECRPA1, LECRPA2, and LECRPA3.

### CROSS-REFERENCE TO RELATED APPLICATIONS

This application claims priority to, and the benefit of the filing date of, U.S. Provisional Patent Application No. 61/787,514, filed on Mar. 15, 2013, entitled “Genetically Modified Plants that are Herbivore-Resistant,” and U.S. Provisional Patent Application No. 61/862,148, filed on Aug. 5, 2013, entitled “Genetically Modified Plants that are Animal-Resistant, Insect-Resistant, and/or Rot-Resistant,” the disclosures of which are incorporated by reference herein in their entireties.

### FIELD OF THE INVENTION

This invention relates to methods for imparting herbivore resistance to plants.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Many plants (timber, agricultural crops, and ornamental plants) are susceptible to damage/consumption by various wildlife (e.g., herbivores). It is estimated that, in the 1990s, wildlife-related economic losses to agricultural crop producers in the United States exceeded $4.5 billion annually (Conover, M. R. 2002. Resolving human-wildlife conflicts: the science of wildlife damage management. Lewis Publishers, Boca Raton, Fla., USA). It is also estimated that annual damage from deer feeding results in $750 million in economic loss in the timber industry and more than $250 million in damage to landscape plantings (Conover, M. R. (1997): Monetary and intangible valuation of deer in the United States. Wildl. Soc. Bull. 25, 298-305). Homeowners also spend an additional $125 million per year on deer damage control. These estimates are conservative, and also outdated because all are based on the statistical data from the 1990s. Deer populations have increased drastically in the 2000s. According to a report from New York's Department of Environmental Conservation, the white-tailed deer population increased from about 500,000 in the early 1900s to 25 million to 30 million nationwide as of 2005 (2005, http://www.msnbc.msn.com/id/6835501/ns/us_news-environment/t/deer-eating-away-forests-nationwide/). Thus, the damage caused by deer, and the resultant negative economic impact, has likely only grown from the 1990s to the present day.

Animal browse causes significant damages to timber, agricultural crops, and ornamental plantings. For the $30 billion the North American timber industry, each year billions of trees are planted in the US and Canada alone. Also, to meet the goal of producing 36 billion gallon of biofuel from biomass by 2022, more poplar and pine trees will be planted in the future. Many of these trees will experience damage by browsing herbivores (such as deer), particularly at early stages of seedling development. Corn, a $60 billion crop, and soybean, a $52 billion crop, are browsed heavily by deer in many regions, causing huge losses to farmers nationwide. Further, ornamental crops and home owners suffer significantly from deer browse.

Unfortunately, currently there are no effective methods available for managing deer and other herbivores that cause huge monetary losses to both plant related industries and the end users.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

As described above, there are numerous problems created by grazing herbivores (such as deer) resulting in large costs to homeowners and industries due to damage to forestry, agricultural crops, and ornamental plants.

However, there are a number of proteins that are toxic to animals and therefore resistant to herbivores. For example, Staphylococcal enterotoxins A(SEA) are protein toxins that, when ingested, cause staphylococcal food poisoning (SFP) syndrome. Initial symptoms, nausea followed by incoercible characteristic vomiting (in spurts), appear 30 min-8 h (3 h on average) after ingesting the contaminated food. Other commonly described symptoms are abdominal pain, diarrhea, dizziness, shivering and general weakness, sometimes associated with a moderate fever. In the most severe cases, prostration and low blood pressure have been reported. In the majority of cases, recovery occurs within 24-48 h without specific treatment, while diarrhea and general weakness can last 24 h or longer. (See Jacques-Antoine Hennekinne, Marie-Laure De Buyser & Sylviane Dragacci. Staphylococcus aureus and its food poisoning toxins: characterization and outbreak investigation. FEMS Microbiol Rev, 2012 (36) 815-836.)

Thus, one aspect of the present invention provides a gene construct including the gene for SEA and a genetically modified plant including this gene. Such plants will then express Staphylococcal enterotoxins A, thereby imparting herbivore resistance to the plant. The SEA proteins may be stored in the endoplasmic reticulum membranes (ERs) or vacuoles of plant leaves, bark, shoot and root tissues.

Further, the black locust tree (which can be found throughout the eastern United States and south-eastern Canada bearing sweet-smelling clusters of white flowers) is resistant to animals (such as herbivores like deer) because it contains toxin proteins called lectins.

Biochemical and molecular studies have demonstrated that black locust tissues, such as bark, express three genes encoding three different lectin polypeptides. One of these genes encodes lectin polypeptide C (26 kDa) that associates exclusively into homotetramers (called Robiniapseudoacacia bark agglutinin II or RPbAII (see vanDamme E J M, Barre A, Smeets K, Torrekens S, van Leuven F, Rouge P, Peumans W J (1995): The bark of Robiniapesudoacacia contains a complex mixture of lectins. Characterization of the proteins and the cDNA clones. Plant Physiol 107:833-843). Two other genes encode lectin polypeptides A and B (31.5 and 29 kDa, respectively), which associate in all possible combinations into five different tetramericisolectins. The mixture of these five isoforms is called Robiniapseudoacacia bark agglutinin I (RPbAI) (Rabijns, A., C. Verboven, P. Rougé, A. Barre, E. J. M. Van Damme, W. J. Peumans and C. J. De Ranter (2001): Proteins Struct. Funct. Genet, 44: 470-478). These three genes may be referred to herein collectively as “RPbAI genes,” and the proteins referred to collectively as “RPbAI proteins.”

As described above, the black locust tree is animal resistant. Thirty minutes after consumption of leaves, barks, young shoots, or seeds of black locust, animals may experience one or more of abdominal pain, acute naseau, diarrhea, vomiting, and cardiac arrhythmia. It is the ingestion of the RPbAI by the animals that causes these symptoms. Such discomfort made experimental animals refuse to continue eating a lectin-containing diet (Rabjins et al., 2001).

Thus, another aspect of the present invention provides for construction of a fusion gene for the three genes from RPbAI (polypeptides A and B) and RPbAII (polypeptide C) from black locust. Another aspect of the present invention provides for a genetically modified plant including these genes. Such plants will then express the toxic lectin proteins, thereby imparting animal (such as deer) resistance to the plant. The proteins may be stored in the endoplasmic reticulum membranes (ERs) or vacuoles of plant leaves, bark, shoot and root tissues.

### BRIEF DESCRIPTION OF THE DRAWINGS

The accompanying drawings, which are incorporated in and constitute a part of this specification, illustrate embodiments of the invention and, together with the general description of the invention given above and the detailed description of the embodiments given below, serve to explain the principles of the present invention.

FIG. 1 is a schematic illustrating a gene construct including the gene for Staphylococcal enterotoxins A.

FIG. 2 is a photograph verifying the transformation of a plant to include the gene for Staphylococcal enterotoxins A.

FIG. 3 is a schematic illustrating a gene construct including the LECRPA1, LECRPA2, and LECRPA3 genes.

FIG. 4 is a photograph verifying the transformation of a plant to include the LECRPA1, LECRPA2, and LECRPA3 genes.

FIG. 5 is a graph showing the verification of expression of the LECRPA3 gene in transgenic plants including the LECRPA1+2+3 construct, using real time PCR.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described above, there are a number of proteins that are toxic to animals and therefore resistant to herbivores. For example, Staphylococcal enterotoxins A(SEA) are protein toxins that, when ingested, cause staphylococcal food poisoning (SFP) syndrome. Initial symptoms, nausea followed by incoercible characteristic vomiting (in spurts), appear 30 min-8 h (3 h on average) after ingesting the contaminated food. Other commonly described symptoms are abdominal pain, diarrhea, dizziness, shivering and general weakness, sometimes associated with a moderate fever. In the most severe cases, prostration and low blood pressure have been reported. In the majority of cases, recovery occurs within 24-48 h without specific treatment, while diarrhea and general weakness can last 24 h or longer. (See Jacques-Antoine Hennekinne, Marie-Laure De Buyser & Sylviane Dragacci. Staphylococcus aureus and its food poisoning toxins: characterization and outbreak investigation. FEMS Microbiol Rev, 2012 (36) 815-836.)

Thus, one aspect of the present invention provides a gene construct including the gene for SEA and a genetically modified plant including this gene. Such plants will then express Staphylococcal enterotoxins A, thereby imparting herbivore resistance to the plant. The SEA proteins may be stored in the endoplasmic reticulum membranes (ERs) or vacuoles of plant leaves, bark, shoot and root tissues.

Further, as described above, the black locust tree (which can be found throughout the eastern United States and south-eastern Canada bearing sweet-smelling clusters of white flowers) is resistant to animals because it contains toxin proteins called lectins.

Biochemical and molecular studies have demonstrated that black locust tissues, such as bark, express three genes encoding three different lectin polypeptides. One of these genes encodes lectin polypeptide C (26 kDa) that associates exclusively into homotetramers (called Robiniapseudoacacia bark agglutinin II or RPbAII (see vanDamme E J M, Barre A, Smeets K, Torrekens S, van Leuven F, Rouge P, Peumans W J (1995): The bark of Robiniapesudoacacia contains a complex mixture of lectins. Characterization of the proteins and the cDNA clones. Plant Physiol 107:833-843). Two other genes encode lectin polypeptides A and B (31.5 and 29 kDa, respectively), which associate in all possible combinations into five different tetramericisolectins. The mixture of these five isoforms is called Robiniapseudoacacia bark agglutinin I (RPbAI) (Rabijns, A., C. Verboven, P. Rouge, A. Barre, E. J. M. Van Damme, W. J. Peumans and C. J. De Ranter (2001): Proteins Struct. Funct. Genet, 44: 470-478).

As described above, it has been determined that certain lectins have potent toxic activity against animals, such as herbivores (e.g., deer). Thus, one aspect of the present invention provides a method for preparing animal-resistant wood, by creating a genetically modified plant (e.g., a woody plant) that expresses proteins encoded by genes such as the RPbAI and RPbAII genes.

As is known to those of ordinary skill in the art, there are multiple ways to obtain the desired DNA sequences for the present invention, and to transform plants therewith. For example, the DNA sequences which code for the SEA proteins of this invention can be obtained by conventional techniques. For example, DNA probes can be constructed to locate the native gene in the genome, and the gene can then be removed by use of appropriate restriction enzymes and spliced into a selected plant expression cassette. Alternatively, an SEA protein can be sequenced in its entirety using known methods, and synthetic DNA sequences can then be prepared which code for the appropriate sequence of amino acids, and this synthetic sequence can be inserted into an appropriate plant expression cassette. These techniques are applicable to each of the genes (e.g., SEA genes) identified herein as useful in this invention.

Further, for example, the DNA sequences which code for the lectin proteins of this invention can be obtained by conventional techniques. For example, DNA probes can be constructed to locate the native gene in the genome, and the gene can then be removed by use of appropriate restriction enzymes and spliced into a selected plant expression cassette. Alternatively, a lectin protein can be sequenced in its entirety using known methods, and synthetic DNA sequences can then be prepared which code for the appropriate sequence of amino acids, and this synthetic sequence can be inserted into an appropriate plant expression cassette. These techniques are applicable to each of the genes (e.g., lectin genes) identified herein as useful in this invention. For example, such techniques may be used with (but not limited to) the LECRPA1, LECRPA2, and/or LECRPA3 genes.

Likewise, numerous plant expression cassettes and vectors are well known in the art. By the term “expression cassette” is meant a complete set of control sequences including initiation, promoter and termination sequences which function in a plant cell when they flank a structural gene in the proper reading frame. Expression cassettes frequently and preferably contain an assortment of restriction sites suitable for cleavage and insertion of any desired structural gene. The cloned gene will have a start codon in the correct reading frame for the structural sequence. In addition, the plant expression cassette preferably includes a strong constitutive promoter sequence at one end to cause the gene to be transcribed at a high frequency, and a poly-A recognition sequence at the other end for proper processing and transport of the messenger RNA. Plant expression cassettes can be designed to include one or more selectable marker genes.

It will also be appreciated by those of ordinary skill that the plant vectors provided herein can be incorporated into Agrobacterium tumefaciens, which can then be used to transfer the vector into susceptible plant cells. Thus, this invention provides a method for imparting herbivore resistance in Agrobacterium tumefaciens-susceptible plants in which the expression cassette is introduced into the cells by infecting the cells with Agrobacterium tumefaciens, a plasmid of which has been modified to include a plant expression cassette (such as is described above). Agrobacterium mediated plant transformation methods are well known to those skilled in the art. However, other methods of transforming plants are well known to those of ordinary skill in the art—and such other methods may be alternatively used to transform plants to impart such herbivore resistance. For example, other aspects of the present invention include a method for imparting herbivore resistance in any plant species using a gene gun or other physical or chemical methods for gene delivery (e.g., that described in Helenius, Elina; Boije, Maria; Niklander-Teeri, Viola; Palva, E. Tapio; Heeri, Teemu U. “Gene Delivery Into Intact Plants Using the Helios Gene Gun”. Plant Molecular Biology Reporter; 2000, 18: 287a-2871, incorporated by reference herein in its entirety).

Thus, one embodiment of the present invention includes cloning and sequencing all cDNA and genomic DNA sequences of the SEA gene or genes. The genomic DNA of the gene should contain promoter, coding and 3′-termination sequences. The cDNA and genomic DNA sequences can then be modified to make them suitable for high expression in leaf, bark, shoot and root organs. Multiple sets of gene constructs may be constructed. For example, one such set may include the SEA gene or genes, using native promoter and 3′-termination sequences. Another such set may use an Alfalfa RbcS gene promoter sequence to control the expression of the coding sequences of the gene. It will be recognized by those of ordinary skill in the art that other genes of interest may be used, as well as other gene promoter sequences (e.g., 35S CaMV gene promoter sequence). An example of such a gene construct is shown schematically in FIG. 1.

In one embodiment, multiple cDNA clones can be isolated from a cDNA library to obtain clones corresponding to SEA polypeptide.

Thus, in various embodiments of the present invention, the gene construct includes SEA. The SEA gene can be incorporated into the tissues of a susceptible plant so that in the course of consuming the plant, the herbivore (e.g., deer) encounters amounts of the selected SEA proteins to cause a sickness in the herbivore. Since the genes which code for these proteins can be isolated, cloned, inserted into an appropriate expression cassette, and introduced into cells of a susceptible plant species, one embodiment of the method involves inserting into the genome of a plant a DNA sequence coding for one or more genes for SEA proteins in proper reading frame relative to transcription initiator and promoter sequences inserted with the genes (or active in the plant). Transcription and translation of the DNA sequence causes expression of the SEA protein sequence at levels which provide a sickness-inducing amount of the SEA protein in the tissues of the plant which are normally consumed by the herbivore (e.g., deer).

The coding sequence for SEA is contained in an open reading frame of 774 base pairs which contains 258 codons, including the translation initiation and termination codons. Analysis of the nucleotide sequence predicts that SEA is synthesized as a protein precursor of 257 amino acid residues (29,700 Mr) which includes a signal sequence of 24 amino acid residues. The hydropathicity curve of the predicted N-terminal amino acids was determined by using the parameters of Kyte and Doolittle. The degree of polarity of the N terminus of SEA is consistent with the structure of known signal sequences; that is, after the initiation Met residue there are two positively charged amino acids which are followed by a stretch of uncharged nonpolar residues. The N-terminal peptide of mature SEA is Ser-Glu-Lys-Ser-Glu-Glu, Therefore, the precursor of SEA is probably processed between Gly-24 and Ser-25, giving a mature protein of 233 residues (27,100 Mr). The predicted amino acid sequence of entA gene agrees well with other data on partial amino acid sequence derived from mature SEA. (See Marsha J. Betley, John J. Mekalanos. Nucleotide Sequence of the Type A Staphylococcal Enterotoxin Gene. Journal of Bacteriology, 1988, 170(1):34-41.)

The nucleic acid sequence of Staphylococcal enterotoxins A(SEA), GenBank No: M18970.1, 774 bp (http://www.ncbi.nlm.nih.gov/nuccore/M18970.1) is:

[SEQ. ID. NO. 1]
ATGAAAAAAACAGCATTTACATTACTTTTATTCATTGCCCTAACGTTGAC
AACAAGTCCACTTGTAAATGGTAGCGAGAAAAGCGAAGAAATAAATGAAA
AAGATTTGCGAAAAAAGTCTGAATTGCAGGGAACAGCTTTAGGCAATCTT
AAACAAATCTATTATTACAATGAAAAAGCTAAAACTGAAAATAAAGAGAG
TCACGATCAATTTTTACAGCATACTATATTGTTTAAAGGCTTTTTTACAG
ATCATTCGTGGTATAACGATTTATTAGTAGATTTTGATTCAAAGGATATT
GTTGATAAATATAAAGGGAAAAAAGTAGACTTGTATGGTGCTTATTATGG
TTATCAATGTGCGGGTGGTACACCAAACAAAACAGCTTGTATGTATGGTG
GTGTAACGTTACATGATAATAATCGATTGACCGAAGAGAAAAAAGTGCCG
ATCAATTTATGGCTAGACGGTAAACAAAATACAGTACCTTTGGAAACGGT
TAAAACGAATAAGAAAAATGTAACTGTTCAGGAGTTGGATCTTCAAGCAA
GACGTTATTTACAGGAAAAATATAATTTATATAACTCTGATGTTTTTGAT
GGGAAGGTTCAGAGGGGATTAATCGTGTTTCATACTTCTACAGAACCTTC
GGTTAATTACGATTTATTTGGTGCTCAAGGACAGTATTCAAATACACTAT
TAAGAATATATAGAGATAATAAAACGATTAACTCTGAAAACATGCATATT
GATATATATTTATATACAAGTTAA

And the amino acid sequence of SEA is:

[SEQ. ID. NO. 2]
MKKTAFTLLLFIALTLTTSPLVNGSEKSEEINEKDLRKKSELQGTALGNL
KQIYYYNEKAKTENKESHDQFLQHTILFKGFFTDHSWYNDLLVDFDSKDI
VDKYKGKKVDLYGAYYGYQCAGGTPNKTACMYGGVTLHDNNRLTEEKKVP
INLWLDGKQNTVPLETVKTNKKNVTVQELDLQARRYLQEKYNLYNSDVFD
GKVQRGLIVFHTSTEPSVNYDLFGAQGQYSNTLLRIYRDNKTINSENMHI
DIYLYTS

Thus, in another particular embodiment, the gene construct may include SEA (shown above), which is then inserted into the genome of a plant in proper reading frame relative to transcription initiator and promoter sequences. Transcription and translation of the DNA sequence causes expression of the SEA protein sequence at levels which provide an amount of the toxin in the tissues of the plant to render the plant resistant to herbivores (such by causing illness in the herbivore). An example of such a gene construct is shown schematically in FIG. 1.

As mentioned above, both genomic and cDNA encoding the gene of interest may be used in this invention. When the gene of interest has been isolated, genetic constructs are made which contain the necessary regulatory sequences to provide for efficient expression of the gene in the host cell. According to this invention, the genetic construct will contain (a) at least one genetic sequence coding for a protein or trait of interest and (b) one or more regulatory sequences operably linked on either side of the structural gene of interest. Typically, the regulatory sequences will be selected from the group comprising of promoters and terminators. The regulatory sequences may be from autologous or heterologous sources.

Thus, in one embodiment, the gene constructs of the present invention also include a gene promoter. As is known to those of ordinary skill in the art a gene promoter is a region of DNA that initiates transcription of a particular gene. Promoters are located near the genes they transcribe, on the same strand and upstream on the DNA (towards the 3′ region of the anti-sense strand). For transcription to take place, RNA polymerase must attach to the DNA near a gene. Promoters contain specific DNA sequences and response elements that provide an initial binding site for RNA polymerase and for transcription factors that recruit RNA polymerase.

In one embodiment, the gene constructs of the present invention may include the Alfalfa RbcS gene promoter RbcSK-1A. Sequence analysis of the promoter RbcSK-1A shows high sequence homology (80%) to the promoter region of the pea RbcS-3A gene. This homology is limited to 235 bp upstream of the first major putative transcription initiation site determined previously. A G-box like sequence found in the promoters of many genes that respond to a variety of different stimuli is also present in the RbcSK-1A promoter. This box binds trans-acting factors which contain a bZIP motif. Twenty-three base pairs downstream of the G-box, another important element in RbcSK-1A promoter region, known as the I-box, was identified. Many light regulated promoters of both monocots and dicots contain this element. [See Khoudi H, Ve'zina L-P, Mercier J, Castonguay Y, Guy A, Laberge S. (1997) An alfalfa rubisco small subunit homologue shares cis acting elements with the regulatory sequence of the RbcS-3A gene from pea. Gene, 197:343-351.]

The nucleic acid sequence of the Alfalfa RbcS gene promoter RbcSK-1A, GenBank: X96847.1, 1803 bp (http://www.ncbi.nlm.nih.gov/nuccore/X96847) is:

[SEQ. ID. NO 3]
TCGACCTGCAGGTCAACGGATCAAATGATTCAATATTTGGCTTGATGAAA
TTAGAGAAAATGAAAAATTGGATTTCTAAGTTTGATTGTTATTTTGAGAT
AGAAAAGGAAAAATCTCTAATCTCTTACGCAAGACCTGCCTCAACCACTT
GATAAACTCTTTTGTCTACGTATTGAAAACAAAAGAGGCAAATAAACATC
TAGCCAAATGAAACACCAATAATGCTTTAAACAAAATGGAATAATTGCAT
CATCAATTAATCTTTATAAGTGAGAATTTTCCCTCCTATAATAATGCGCT
AGGTATCAATTTTCAACTCTGAAATATAAAGCTTCAAGCGTGTGTTATCA
AAAATCAAGCACAGTAAAATCATAAGCAGAATCATTGGTGATGCTAATAG
TTGATGTGGAATCGAACAATGTTCATATTCTGATACCTTGTTGGTGACAA
GTCAAGACCCTTATAGACTTGAATTTTGTCTGAGTTGATGATTTCAGAAG
GGGAATCTAGTATCTAAGTAGATGGTAAATTTATTTTTTCCAATTCCAGT
TGCTTCCATTATGAACAAACCTTATTCTTTTAGGCTAATATTGAGGAACA
AAAGCCACGGAATATTTTTTTTATGTATAACCTAAGAAAAAGACAATAAT
AAAAATAATTAAAAACTACAACAGATGATTTTGGACTTGAATCGAATTGG
ATTAATCTTATACATGTTGTCGATAAGGATACTAGTTATATGAAGAAGAG
AATCAATTGAAACTTTATTTGTGCTATATATAATGATTTATGATATATGG
AGAGAGGGATGGCAGAATATGCAAGTTTGGAATCAATTCTGGACATTCAT
GGAGGGCGGGTTTATCATCGTGGGTGTGGTAGGGGTGGCTGAGGTTCTAG
GGCTACCCAGTACTTTTATGATGTTGTTGTAGTATTTTGAACAAATTTGT
TTTTTAATTTTATGTTTGAATATTGGGTTTGTAACATATGGATAATTTGT
TTTTAATTTTATGTTCGAAAATTGTGTTGTTTTTGATCATTTTCATTACA
ATATTTATTTATTTATTCACGAATGCATGTTTATATCAACAAATTATATA
ATCTGTATGTATCATAGTGAAAACAAACTCTGTTTTTCTTTTGATACCTT
TCAGATTATATAATTTGAAATGTCATAAAACAGTTTAGATTATATAATCT
GAAATATGCGTTTTTTACACCACATTTTGCATTTTGTGAGGTGTTTGACA
CTTTTCGGATTATATAAGTCAAATTATTTTAAGAAGTTTCGGATTATATA
TCTGAAACATATGTTTAACTGACACATACACAAACATCTCTAGGGTGATT
TGTCTTCCAATAGTTTTTATACTGTTTGGATTATATAATCCGAATCAAGG
TTAAGAAAAAATTAGGGCGCTCGAAAACCAAATAGGGTGGGAAAAGTAAT
GACCAATATTGATTACCCTATAAGGAGCCAAAGCCTGAAAAAAGTACCAT
ACATGATTGATATTTGTGGAGGCATTAATAGTCACAAAACTACACGTGGC
AATTTTATATTGGTGGCTAATGATAAGGCTAGCACAAAAATTTCCATTCC
TGTGTGGTTGATATGGCAGCAAAGTTTATCATATTCACAACCAACAAAAT
GGTATTATGAAGCATTACCACAATTTATAAGACCATAATATTGGAAATAG
GAAAATAAAAACATTATATATAGCAAGTTTGAGTATAAGCTTTGCAATTC
AAGCAGAAGTACATCTTACTTTACTAGTGAACTAAGTAAGGGAGAAAAAA
AATGGCTTCCTCTATGATGTCCTCTTCA

The gene constructs of the present invention also include Rubisco Small Subunit Transit Peptide (RS), which increases the gene expression in chloroplasts. Rubisco Small Subunit Transit Peptide can increase the accumulation of protein in chloroplasts of transgenic plant. The majority of chloroplast proteins are encoded in the nucleus and synthesized in the cytosol as precursors with N-terminal extensions called transit peptides. The N-terminal transit peptide generally possesses necessary and sufficient information for the correct targeting of proteins to chloroplasts. Suyeon found that RS:CeI5A transgenic lines produced highly stable active enzymes, and the protein accumulation of these transgenic lines was up to 5.2% of the total soluble protein in the crude leaf extract, remaining stable throughout the life cycle of the tobacco plant. (See Suyeon Kim, Dae-Seok Lee, In Seong Choi, Sung-Ju Ahn, Yong-Hwan Kim, Hyeun-Jong Bae, Arabidopsis thaliana Rubisco small subunit transit peptide increases the accumulation of Thermotoga maritime endoglucanase CeI5A in chloroplasts of transgenic tobacco plants. Transgenic Res. 2010, (19):489-497.)

The nucleic acid sequence of Rubisco Small Subunit Transit Peptide (RS) (http://www.nbi.nlm.nih.gov/nucleotide/145362366?report=genbank&log$=nucialign&bl ast_rank=1&RID=KHUF0DCR016) is:

[SEQ. ID. NO. 4]
ATGGCTTCCTCTATGCTCTCTTCCGCTACTATGGTTGCCTCTCCGGCTCA
GGCCACTATGGTCGCTCCTTTCAACGGACTTAAGTCCTCCGCTGCCTTCC
CAGCCACCCGCAAGGCTAACAACGACATTACTTCCATCACAAGCAACGGC
GGAAGAGTTAACTGCATGCAGGTGTGGCCTCCGATTGGAAAGAAGAAGTT
TGAGACTCTCTCTTACCTTCCTGACCTTACCGATTCCGAA

And the amino acid sequence is:

[SEQ. ID. NO. 5]
MASSMLSSATMVASPAQATMVAPFNGLKSSAAFPATRKANNDITSITSNG
GRVNCMQVWPPIGKKKFETLSYLPDLTDSE

The above regulatory sequences may be used in the gene constructs for herbivore resistance.

Another embodiment of the present invention includes cloning and sequencing all cDNA and genomic DNA sequences of the three RPbAI/RPbAII genes from black locust. The genomic DNA of the three genes should contain their promoter, coding and 3′-termination sequences. The cDNA and genomic DNA sequences can then be modified to make them suitable for high expression in leaf, bark, shoot and root organs. Multiple sets of fusion genes may be constructed. For example, one such set may include the three native black locust genes, using their native promoter and 3′-termination sequences. Another such set may use an Alfalfa RbcS gene promoter sequence to control the expression of the coding sequences of the three black locust gene. It will be recognized by those of ordinary skill in the art that other genes of interest may be used, as well as other gene promoter sequences (e.g., 35S CaMV gene promoter sequence). An example of such a fusion gene construct is shown schematically in FIG. 3.

In one embodiment, multiple cDNA clones can be isolated from a cDNA library to obtain three classes of lectin cDNA clones corresponding to the a and b polypeptides of RPbAI and the c polypeptide of RPbAII. These cDNA clones are referred to as LECRPA1, LECRPA2, and LECRPA3.

Thus, in various embodiments of the present invention, the gene construct includes LECRPA1, LECRPA2, or LECRPA3, or any combination of such lectins. The lectin can be incorporated into the tissues of a susceptible plant so that in the course of infesting the plant, the animal (e.g., deer) encounters animal-toxic amounts of the selected lectin or lectins. Since the genes which code for these lectins can be isolated, cloned, inserted into an appropriate expression cassette, and introduced into cells of a susceptible plant species, one embodiment of the method involves inserting into the genome of a plant a DNA sequence coding for one or more animal-toxic plant lectins in proper reading frame relative to transcription initiator and promoter sequences inserted with the genes (or active in the plant). Transcription and translation of the DNA sequence causes expression of the lectin protein sequence at levels which provide an animal-toxic amount of the lectin in the tissues of the plant which are normally infested or consumed by the animal (e.g., deer).

LECRPA1, contains an 856-bp open reading frame encoding a 285-amino acid precursor with one possible initiation codon at position 1 of the deduced amino acid sequence. Translation starting with this Met residue results in a lectin precursor with a calculated molecular mass of 30,928 D, which after co-translational cleavage of the signal peptide of 31 amino acids yields a lectin precursor polypeptide of 27,330 D with an N-terminal amino acid sequence identical to the one determined for the a polypeptide of RPbAI.

The estimated pl for the lectin polypeptide encoded by LECRPA1 is 5.04. The deduced amino acid sequence of the lectin cDNA clone LECRPA1 contains two putative glycosylation sites at positions 147 and 188. (See Van Damme E J M, Barre A, Smeets K, Torrekens S, Van Leuven F, Rouge P, Peumans W J (1995b) The bark of Robinia pseudoacacia contains a complex mixture of lectins. Plant Physiol 107:833-843, incorporated by reference herein in its entirety.)

The following is the gene sequence of LECRPA1 [GenBank: U12782.1, 858 bp (http://www.ncbi.nlm.nih.gov/nuccore/U12782.1)]:

[SEQ. ID. NO. 6]
ATGACTTCCTACAACTTCAAAACCCAAACCTCCTTCCCTCTTCTCCTATC
CATATCCTTTTTTTTCCTCTTGTTACTCAACAAGGTGAATTCAACTGGAT
CTCTCTCCTTTTCTTTCCCCAAGTTCGCGCCTAACCAACCATATCTGATC
TTCCAACGTGATGCCCTTGTGACATCAACAGGGGTGTTACAACTCACCAA
CGTAGTTAACGGGGTACCATCCGGTAAATCTCTTGGTAGAGCTCTATATG
CTGCCCCTTTCCAAATCTGGGATAGCACCACAGGCAACGTGGCTAGCTTT
GTCACTTCCTTCTCCTTTATCATTCAAGCACCTAACCCAACCACAACGGC
AGATGGTCTTGCCTTCTTTCTTGCACCAGTTGATACTCAGCCCTTAGATG
TTGGAGGAATGCTCGGAATTTTCAAAGACGGATATTTCAATAAATCCAAC
CAAATTGTTGCAGTTGAATTCGATACCTTTTCAAATATTCACTTTGATCC
AAAAGGTAGACATATGGGAATCAATGTCAACTCCATCGTGTCCATAAAAA
CCGTGCCATGGAATTGGACAAATGGCGAAGTAGCCAATGTTTTCATAAGC
TATGAAGCTTCCACCAAATCCTTAACTGCCTCTTTGGTTTATCCTTCACT
TGAAACAAGTTTTATCGTTCATGCTATTGTGGATGTGAAGGATGTTCTTC
CCGAGTGGGTAAGATTTGGTTTCTCAGCTACCACAGGAATAGATAAAGGC
TACGTTCAAACAAATGATGTTCTCTCCTGGTCTTTCGAGTCAAACTTGCC
AGGTGGTAACAGTGTTGCTTCGGTGAAGAACGCGGGTCTTTCAACCTATG
CTGCATGA.

And the amino acid sequence of LECRPA1 is:

[SEQ. ID. NO. 7]
MTSYNFKTQTSFPLLLSISFFFLLLLNKVNSTGSLSFSFPKFAPNQPYLI
FQRDALVTSTGVLQLTNVVNGVPSGKSLGRALYAAPFQIWDSTTGNVASF
VTSFSFIIQAPNPTTTADGLAFFLAPVDTQPLDVGGMLGIFKDGYFNKSN
QIVAVEFDTFSNIHFDPKGRHMGINVNSIVSIKTVPWNWTNGEVANVFIS
YEASTKSLTASLVYPSLETSFIVHAIVDVKDVLPEWVRFGFSATTGIDKG
YVQTNDVLSWSFESNLPGGNSVASVKNAGLSTYAA

LECRPA2 encodes a 286-amino acid precursor with a calculated molecular mass of 31,211 D, which after cleavage of the signal peptide, 31 amino acids, is converted into a 27,600 D lectin polypeptide with an N-terminal amino acid sequence similar to the one determined for the b polypeptide of RPbAI and an estimated pl of 4.95. The sequence of this lectin polypeptide contains only one putative N-glycosylation site, the position of which coincides with the first possible glycosylation site in LECRPA1. (See Van Damme E J M, Barre A, Smeets K, Torrekens S, Van Leuven F, Rouge P, Peumans W J (1995b) The bark of Robinia pseudoacacia contains a complex mixture of lectins. Plant Physiol 107:833-843.)

The nucleic acid sequence of LECRPA2 [GenBank: U12783.1, 861 bp (http://www.ncbi.nlm.nih.gov/nuccore/U12783)] is:

[SEQ. ID. NO. 8]
ATGGCTTCCTACAAGTTCAAAACCCAAAACTCCTTCCTTCTTCTCCTATC
CATATCCTTTTTCTTCCTCTTGTTACTCAACAAGGTGAATTCGACTGGAT
CCCTCTCCTTTTCTTTCCCCAAGTTCAAGCATAGCCAACCAGATCTGATC
TTCCAAAGTGATGCCCTTGTGACATCAAAAGGGGTGTTACAACTCACCAC
GGTAAATGATGGAAGACCAGTCTATGACTCTATTGGTCGAGTTCTATATG
CTGCCCCTTTCCAAATTTGGGATAGCACCACTGGCAACGTGGCTAGCTTT
GTCACTTCCTTCTCCTTTATCATCAAAGCACCTAACGAAGGCAAAACGGC
AGATGGTCTTGTCTTCTTTCTTGCACCAGTTGGTAGTACTCAGCCCCTAA
AAGGAGGAGGACTCCTCGGACTTTTCAAAGATGAATCTTACAATAAATCC
AACCAAATTGTTGCAGTTGAATTTGACACATTTCGGAATGTTGCATGGGA
TCCAAATGGAATACATATGGGAATCGATGTCAACTCTATTCAATCCGTAA
GAACTGTGCGATGGGATTGGGCGAATGGCGAAGTAGCCAATGTTTTCATA
AGCTATGAAGCTTCCACCAAATCCTTAACTGCCTCTTTGGTTTATCCTTC
ACTTGAAAAAAGTTTTATCTTGAGTGCTATTGTGGATTTGAAGAAAGTTC
TTCCGGAGTGGGTAAGAGTTGGTTTCACAGCTACCACAGGACTATCTGAA
GACTACGTTCAAACAAATGATGTTCTCTCCTGGTCTTTCGAGTCAAACTT
GCCAGGTGGTAACAGTGTTGCTTCGGTGAAGAACGCGGGTCTTTCAACCT
ATGCTGCATGA

And the amino acid sequence of LECRPA2 is:

[SEQ. ID. NO. 9]
MASYKFKTQNSFLLLLSISFFFLLLLNKVNSTGSLSFSFPKFKHSQPDLI
FQSDALVTSKGVLQLTTVNDGRPVYDSIGRVLYAAPFQIWDSTTGNVASF
VTSFSFIIKAPNEGKTADGLVFFLAPVGSTQPLKGGGLLGLFKDESYNKS
NQIVAVEFDTFRNVAWDPNGIHMGIDVNSIQSVRTVRWDWANGEVANVFI
SYEASTKSLTASLVYPSLEKSFILSAIVDLKKVLPEWVRVGFTATTGLSE
DYVQTNDVLSWSFESNLPGGNSVASVKNAGLSTYAA

LECRPA3, encodes a 272-amino acid precursor with one possible initiation site at position 13. Translation starting at this site yields a lectin precursor with a calculated molecular mass of 27,878 D, which after cleavage of the signal peptide, 17 amino acids, is converted into a 25,970 D lectin polypeptide, the N-terminal sequence of which resembles the sequence determined for the c polypeptide of RPbAII.

The estimated pl of the polypeptide encoded by LECRPA3 is 6.5, higher than the pl of the lectin polypeptides encoded by LECRPA1 and LECRPA2. Within the coding sequence of LECRPA3, three putative glycosylation sites are present at positions 36, 39, and 65 of the lectin precursor. (See Van Damme E J M, Barre A, Smeets K, Torrekens S, Van Leuven F, Rouge P, Peumans W J (1995b) The bark of Robinia pseudoacacia contains a complex mixture of lectins. Plant Physiol 107:833-843.)

The nucleic acid sequence of LECRPA3 [GenBank: U12784.1, 783 bp (http://www.ncbi.nlm.nih.gov/nuccore/U12784)] is:

[SEQ. ID. NO. 10]
ATGCTCATAAGTTTCTTTGTCTTGCTAGCTAGTGCCAGAAAGGAGAACTC
TGATGAAGGAATTTCCTTCAACTTCACCAACTTCACCAGAGGTGATCAAG
GTGTAACCTTACTAGGACAAGCCAACATTATGGCAAATGGGATCTTGGCC
CTCACCAACCATACAAACCCTACTTGGAATACAGGCCGTGCCTTGTATTC
TAAACCAGTTCCTATTTGGGATTCAGCCACTGGCAATGTCGCCAGCTTTG
TTACTTCCTTCTCTTTTGTCGTACAAGAGATCAAAGGTGCTATACCAGCT
GATGGAATTGTTTTCTTCCTTGCACCAGAAGCCAGGATTCCCGACAATTC
AGCCGGTGGGCAACTCGGAATTGTTAATGCCAACAAAGCTTACAATCCAT
TTGTTGGTGTAGAATTTGATACTTACTCCAATAATTGGGATCCTAAATCT
GCACATATTGGAATCGATGCCAGCTCTTTAATTTCATTAAGGACTGTGAA
ATGGAACAAGGTTAGTGGGTCATTGGTCAAAGTTAGTATCATCTATGACT
CTCTATCTAAGACGTTGAGTGTTGTTGTGACTCACGAGAATGGTCAAATT
TCTACCATCGCTCAAGTCGTGGATTTGAAAGCTGTGCTGGGAGAGAAGGT
CAGGGTTGGTTTTACTGCAGCCACCACAACAGGCCGGGAATTATACGACA
TTCATGCATGGTCTTTCACTTCAACTTTGGTGACAGCTACAAGCAGCACC
TCGAAGAACATGAATATTGCAAGCTATGCATGA

And the amino acid sequence of LECRPA3 is:

[SEQ. ID. NO. 11]
MLISFFVLLASARKENSDEGISFNFTNFTRGDQGVTLLGQANIMANGILA
LTNHTNPTWNTGRALYSKPVPIWDSATGNVASFVTSFSFVVQEIKGAIPA
DGIVFFLAPEARIPDNSAGGQLGIVNANKAYNPFVGVEFDTYSNNWDPKS
AHIGIDASSLISLRTVKWNKVSGSLVKVSIIYDSLSKTLSVVVTHENGQI
STIAQVVDLKAVLGEKVRVGFTAATTTGRELYDIHAWSFTSTLVTATSST
SKNMNIASYA

Thus, in one particular embodiment, the gene construct may include LECRPA1, LECRPA2, and LECRPA3 (shown above), which is then inserted into the genome of a plant in proper reading frame relative to transcription initiator and promoter sequences. Transcription and translation of the DNA sequence causes expression of the lectin protein sequence at levels which provide an animal-toxic (e.g., naseau or sickness-inducing) amount of the lectin in the tissues of the plant which are normally infested or consumed by the animal (e.g., deer).

As mentioned above, both genomic and cDNA encoding the gene of interest may be used in this invention. When the gene of interest has been isolated, genetic constructs are made which contain the necessary regulatory sequences to provide for efficient expression of the gene in the host cell. According to this invention, the genetic construct will contain (a) at least one genetic sequence coding for a protein or trait of interest and (b) one or more regulatory sequences operably linked on either side of the structural gene of interest. Typically, the regulatory sequences will be selected from the group comprising of promoters and terminators. The regulatory sequences may be from autologous or heterologous sources.

Thus, in one embodiment, the gene constructs of the present invention also include a gene promoter. As is known to those of ordinary skill in the art a gene promoter is a region of DNA that initiates transcription of a particular gene. Promoters are located near the genes they transcribe, on the same strand and upstream on the DNA (towards the 3′ region of the anti-sense strand). For transcription to take place, RNA polymerase must attach to the DNA near a gene. Promoters contain specific DNA sequences and response elements that provide an initial binding site for RNA polymerase and for transcription factors that recruit RNA polymerase.

In one embodiment, the gene constructs of the present invention may include the Alfalfa RbcS gene promoter RbcSK-1A. Sequence analysis of the promoter RbcSK-1A shows high sequence homology (80%) to the promoter region of the pea RbcS-3A gene. This homology is limited to 235 bp upstream of the first major putative transcription initiation site determined previously. A G-box like sequence found in the promoters of many genes that respond to a variety of different stimuli is also present in the RbcSK-1A promoter. This box binds trans-acting factors which contain a bZIP motif. Twenty-three base pairs downstream of the G-box, another important element in RbcSK-1A promoter region, known as the I-box, was identified. Many light regulated promoters of both monocots and dicots contain this element. [See Khoudi H, Ve'zina L-P, Mercier J, Castonguay Y, Guy A, Laberge S. (1997) An alfalfa rubisco small subunit homologue shares cis acting elements with the regulatory sequence of the RbcS-3A gene from pea. Gene, 197:343-351, incorporated by reference herein in its entirety.]

The nucleic acid sequence of the Alfalfa RbcS gene promoter RbcSK-1A, GenBank: X96847.1, 1803 bp (http://www.ncbi.nlm.nih.gov/nuccore/X96847) is shown above, and is:

[SEQ. ID. NO 3]
TCGACCTGCAGGTCAACGGATCAAATGATTCAATATTTGGCTTGATGAAA
TTAGAGAAAATGAAAAATTGGATTTCTAAGTTTGATTGTTATTTTGAGAT
AGAAAAGGAAAAATCTCTAATCTCTTACGCAAGACCTGCCTCAACCACTT
GATAAACTCTTTTGTCTACGTATTGAAAACAAAAGAGGCAAATAAACATC
TAGCCAAATGAAACACCAATAATGCTTTAAACAAAATGGAATAATTGCAT
CATCAATTAATCTTTATAAGTGAGAATTTTCCCTCCTATAATAATGCGCT
AGGTATCAATTTTCAACTCTGAAATATAAAGCTTCAAGCGTGTGTTATCA
AAAATCAAGCACAGTAAAATCATAAGCAGAATCATTGGTGATGCTAATAG
TTGATGTGGAATCGAACAATGTTCATATTCTGATACCTTGTTGGTGACAA
GTCAAGACCCTTATAGACTTGAATTTTGTCTGAGTTGATGATTTCAGAAG
GGGAATCTAGTATCTAAGTAGATGGTAAATTTATTTTTTCCAATTCCAGT
TGCTTCCATTATGAACAAACCTTATTCTTTTAGGCTAATATTGAGGAACA
AAAGCCACGGAATATTTTTTTTATGTATAACCTAAGAAAAAGACAATAAT
AAAAATAATTAAAAACTACAACAGATGATTTTGGACTTGAATCGAATTGG
ATTAATCTTATACATGTTGTCGATAAGGATACTAGTTATATGAAGAAGAG
AATCAATTGAAACTTTATTTGTGCTATATATAATGATTTATGATATATGG
AGAGAGGGATGGCAGAATATGCAAGTTTGGAATCAATTCTGGACATTCAT
GGAGGGCGGGTTTATCATCGTGGGTGTGGTAGGGGTGGCTGAGGTTCTAG
GGCTACCCAGTACTTTTATGATGTTGTTGTAGTATTTTGAACAAATTTGT
TTTTTAATTTTATGTTTGAATATTGGGTTTGTAACATATGGATAATTTGT
TTTTAATTTTATGTTCGAAAATTGTGTTGTTTTTGATCATTTTCATTACA
ATATTTATTTATTTATTCACGAATGCATGTTTATATCAACAAATTATATA
ATCTGTATGTATCATAGTGAAAACAAACTCTGTTTTTCTTTTGATACCTT
TCAGATTATATAATTTGAAATGTCATAAAACAGTTTAGATTATATAATCT
GAAATATGCGTTTTTTACACCACATTTTGCATTTTGTGAGGTGTTTGACA
CTTTTCGGATTATATAAGTCAAATTATTTTAAGAAGTTTCGGATTATATA
TCTGAAACATATGTTTAACTGACACATACACAAACATCTCTAGGGTGATT
TGTCTTCCAATAGTTTTTATACTGTTTGGATTATATAATCCGAATCAAGG
TTAAGAAAAAATTAGGGCGCTCGAAAACCAAATAGGGTGGGAAAAGTAAT
GACCAATATTGATTACCCTATAAGGAGCCAAAGCCTGAAAAAAGTACCAT
ACATGATTGATATTTGTGGAGGCATTAATAGTCACAAAACTACACGTGGC
AATTTTATATTGGTGGCTAATGATAAGGCTAGCACAAAAATTTCCATTCC
TGTGTGGTTGATATGGCAGCAAAGTTTATCATATTCACAACCAACAAAAT
GGTATTATGAAGCATTACCACAATTTATAAGACCATAATATTGGAAATAG
GAAAATAAAAACATTATATATAGCAAGTTTGAGTATAAGCTTTGCAATTC
AAGCAGAAGTACATCTTACTTTACTAGTGAACTAAGTAAGGGAGAAAAAA
AATGGCTTCCTCTATGATGTCCTCTTCA

The gene constructs of these embodiments of the present invention also include Rubisco Small Subunit Transit Peptide (RS), which increases the gene expression in chloroplasts. Rubisco Small Subunit Transit Peptide can increase the accumulation of protein in chloroplasts of transgenic plant. The majority of chloroplast proteins are encoded in the nucleus and synthesized in the cytosol as precursors with N-terminal extensions called transit peptides. The N-terminal transit peptide generally possesses necessary and sufficient information for the correct targeting of proteins to chloroplasts. Suyeon found that RS:CeI5A transgenic lines produced highly stable active enzymes, and the protein accumulation of these transgenic lines was up to 5.2% of the total soluble protein in the crude leaf extract, remaining stable throughout the life cycle of the tobacco plant. (See Suyeon Kim, Dae-Seok Lee, In Seong Choi, Sung-Ju Ahn, Yong-Hwan Kim, Hyeun-Jong Bae, Arabidopsis thaliana Rubisco small subunit transit peptide increases the accumulation of Thermotoga maritime endoglucanase CeI5A in chloroplasts of transgenic tobacco plants. Transgenic Res. 2010, (19):489-497, incorporated by reference herein in its entirety.)

The nucleic acid sequence of Rubisco Small Subunit Transit Peptide (RS) (http://www.ncbi.nlm.nih.gov/nucleotide/145362366?report=genbank&log$=nuclalign&bl ast_rank=1&RID=KHUF0DCR016) is shown above, and is:

[SEQ. ID. NO. 4]
ATGGCTTCCTCTATGCTCTCTTCCGCTACTATGGTTGCCTCTCCGGCTCA
GGCCACTATGGTCGCTCCTTTCAACGGACTTAAGTCCTCCGCTGCCTTCC
CAGCCACCCGCAAGGCTAACAACGACATTACTTCCATCACAAGCAACGGC
GGAAGAGTTAACTGCATGCAGGTGTGGCCTCCGATTGGAAAGAAGAAGTT
TGAGACTCTCTCTTACCTTCCTGACCTTACCGATTCCGAA

And the amino acid sequence is shown above, and is:

[SEQ. ID. NO. 5]
MASSMLSSATMVASPAQATMVAPFNGLKSSAAFPATRKANNDITSITSNG
GRVNCMQVWPPIGKKKFETLSYLPDLTDSE

The above regulatory sequences may be used in the gene constructs for animal resistance. They may be used to create such gene constructs (and the expression cassettes described above) using techniques that are known to those skilled in the art.

In one embodiment, the plant to be transformed in aspects of the present invention is preferably a plant susceptible to damage by herbivores. Such plants include Arabidopsis and Canola. However, this is not to be construed as limiting to these species. Thus the methods of this invention are readily applicable via conventional techniques to numerous plant species, if they are found to be susceptible to the plant pests listed hereinabove.

Further, while plants, and transformed or transgenic plants are discussed herein, the term “plant” may include the whole plant or any parts or derivatives thereof, such as plant cells, plant protoplasts, plant cell tissue cultures from which plants can be regenerated, plant calli, plant cell clumps, and plant cells that are intact in plants, or parts of plants, such as embryos, pollen, ovules, fruit, flowers, leaves, seeds, roots, root tips, and the like.

Further still, while various sequences of genes are described herein (such as sequences for LECRPA1, LECRPA2, and LECRPA3), it will be recognized by those skilled in the art that exact sequences need not be used in the transgenic plants (and other aspects of the present invention), so long as the sequence used is functional for its intended purpose (e.g., expression thereof provides resistance to an herbivore). Thus, aspects of the present invention contemplate variants of the genes and sequences described herein. As used herein, the term “variant” refers to nucleic acid sequences that are essentially similar to a given nucleic acid sequence. For example, the term “variants thereof” or refers to a polynucleotide sequence having one or more (e.g., two, three, four, five or more) nucleotides deleted (deletion variants) from said polynucleotide sequence or having one or more nucleotides substituted (substitution variants) with other nucleotides or one or more nucleotides inserted into said polynucleotide sequence (insertion variants). Sequences which are essentially similar to one another are nucleic acid sequences comprising at least about 90%, more preferably 91%, 92%, 93%, 94%, 95%, 96%, 97%, 98% or 99% or more nucleic acid sequence identity to one or more listed sequences.

In one illustrative embodiment, the DNA sequence, which when expressed imparts a sickness-inducing activity, may a structural gene which codes for at least one SEA protein as described herein. Alternatively, there may be used a construct of multiple genes coding for more than one SEA protein. Further, it will be recognized by those skilled in the art that other proteins may exhibit some sickness-inducing activity to herbivores. It would be within the purview of one skilled in the art to prepare gene constructs and transformed plants including such other genes based on the teachings herein.

Alternatively, the DNA sequence, which when expressed imparts and animal-sickness-inducing activity, is a structural gene which codes for at least one of the selected plant lectins described herein. Alternatively, there may be used a construct of multiple genes coding for more than one of the lectins, such as for all three above-described lectins. Further, it will be recognized by those skilled in the art that other lectins may exhibit some animal-sickness-inducing activity. Further, other proteins beside lectins may exhibit animal-sickness-inducing activity. It would be within the purview of one skilled in the art to prepare gene constructs and transformed plants including such constructs based on the teachings herein.

Further, in general, since one aspect of the invention is to confer resistance to an herbivore to which the plant is susceptible, a selected protein will not be native to the plant (in any case where such a protein may be a native plant protein), i.e., the protein will come from a species other than the plant being transformed. However, in any plant species which produce such proteins, but in lower than sickness-inducing amounts, it may be preferable to insert a gene for the native protein under strong constitutive promoter control to cause overproduction of the protein, thus achieving sickness-inducing levels and conferring effective resistance. Alternatively, where a plant produces a native protein, but the protein is not produced in or not distributed to tissues which are normally consumed, a tissue specific promoter can be used to provide localized expression or overproduction of the protein. A tissue specific promoter can be used in any instance where it may be desirable to localize production of the protein to an consumable tissue or to a tissue which is efficient in production of the protein.

In one embodiment of the present invention, transgenic Arabidopsis is prepared with above-described genes using an Agrobacterium mediated plant transformation method. Arabidopsis is a relative of cabbage and canola. Arabidopsis is thus an excellent model plant to test the effectiveness of the proposed strategy in creating animal resistant plants. In another embodiment of the present invention, transgenic Canola may be prepared with above-described genes using techniques such as those described above, or other methods known to those skilled in the art (e.g., Agrobacterium mediated plant transformation, gene gun, or other physical or chemical methods for gene delivery).

Once transgenic plants are produced, a method of the present invention includes selecting those with high expression levels of the desired genes. Such transgenic plants with high expression levels of the desired genes may then be used for the desired resistance traits. Also, further experiments may be performed on such plants to determine the effectiveness of the resistance traits.

Further, it is possible that expression levels of the engineered SEA genes may not be high enough in leaf, bark, flower or root tissues to provide effective resistance. If that is the case, additional DNA sequences may be inserted into the proposed fusion genes to enhance the transcription and translation of the SEA genes (via techniques well known to those skilled in the art, and as described above). A vacuole localization signal peptide may also be used to produce higher accumulation levels of the SEA proteins in vacuoles of leaf, bark and shoot tissues.

Thus, the present invention contemplates: (A) molecular cloning and modifications of the SEA gene, and construction of fusion genes for high expression in plants; (B) genetic transformation of plants (such as Aribdopsis or Canola); (C) characterization of expression levels of transgenes in transgenic plants; (D) propagation of selected lines of transgenic plants; and (E) use of such plants for their resistance to herbivores.

Further, a recent patent and other research articles report the use of RPbAI to treat diseases of the gastrointestinal tract including mucositis (US patent no. PCT/GB98/02612). Since medical applications of RPbAI require large quantities of inexpensive, high quality RPbAI protein a transgenic plant source could be of commercial significance.

Further, the present invention contemplates: (A) molecular cloning and modifications of the three RPbAI genes, and construction of fusion genes for high expression in plants; (B) genetic transformation of plants (such as Aribdopsis or Canola); (C) characterization of expression levels of transgenes in transgenic plants; (D) propagation of selected lines of transgenic plants; and (E) use of such plants for their resistance to herbivores.

The following Examples further exemplify the gene constructs and transgenic plants of the various aspects of this invention and the methods of making and using them. However, it will be understood that other methods, known by those of ordinary skill in the art to be equivalent, can also be employed.

### EXAMPLES

### Example 1

### Transformation of Plants

In this Example, transgenic Arabidopsis is prepared with above-described genes using an Agrobacterium mediated plant transformation method. It will be noted that not all plants described in the various Examples below were transformed using this method, and other methods for transforming plants are well know, and have been described elsewhere in this application.

As is known to those skilled in the art, Agrobacterium is a genus of Gram-negative bacteria that uses horizontal gene transfer to cause tumors in plants. Transformation with Agrobacterium can be achieved in two ways. Protoplasts, or leaf-discs can be incubated with the Agrobacterium and whole plants regenerated using plant tissue culture. A common transformation protocol for Arabidopsis is the floral-dip method: the flowers are dipped in an Agrobacterium culture, and the bacterium transforms the germline cells that make the female gametes. The seeds can then be screened for antibiotic resistance (or another marker of interest), and plants that have not integrated the plasmid DNA will die.

Agrobacterium tumefaciens is the most commonly studied species in this genus and infects the plant through its Ti plasmid. The Ti plasmid integrates a segment of its DNA, known as T-DNA, into the chromosomal DNA of its host plant cells. The plasmid T-DNA that is transferred to the plant is an ideal vehicle for genetic engineering. This is done by cloning a desired gene sequence into the T-DNA that will be inserted into the host DNA (and so gene constructs for LECRPA1, 2, and 3 may be inserted in this fashion). Arabidopsis may be transformed by dipping their flowers into a broth of Agrobacterium: the seed produced will be transgenic.

Floral Dip Transformation of Arabidopsis

Plants were typically planted 6-20 per 64 cm² pot in moistened potting soil. To obtain more floral buds per plant, inflorescences were clipped after most plants had formed primary bolts, relieving apical dominance and encouraging synchronized emergence of multiple secondary bolts. Plants were dipped when most secondary inflorescences were about 1-10 cm tall.

Inoculation of Plants

Agrobacterium tumefaciens cultures were typically started from a 1:100 dilution of smaller overnight cultures and grown for roughly 18-24 h. Cells were harvested by centrifugation for 20 min at room temperature at 5500 g and then re-suspended in infiltration medium to a final OD600 of approximately 0.80 prior to use. The revised floral dip inoculation medium contained 5.0% sucrose and 0.05% Silwet L-77. For floral dip, the inoculum was added to a beaker, plants were inverted into this suspension such that all above ground tissues were submerged, and plants were then removed after 3-5 sec of gentle agitation. Plants were left in a low light or dark location overnight and returned to the greenhouse the next day. Plants were grown for a further 3-5 weeks until siliques were brown and dry.

Selection of Putative Transformants

Seeds were surface sterilized by liquid sterilization, seeds were first treated with 95% ethanol for 30-60 sec, then with 50% bleach containing 0.05% Tween 20 for 5 min, followed by three rinses with sterile water. To select for transformed plants, sterilized seeds were suspended in 0.1% sterile agarose and plated on kanamycin selection plates at a density of approximately 3000 seeds per 150*15 mm2 plate, cold-treated for 2 days, and then grown for 7-10 days in a controlled environment at 24° C. Selection plates contained ½×MS medium, 0.8% agar, 50 mg ml-1 kanamycin mono sulfate. Petri plates and lids were sealed with surgical tape for the first week of growth. Excess moisture during growth was removed by briefly opening the plates and shaking moisture off the lid. Transformants were identified as kanamycin resistant seedlings that produced green leaves and well established roots within the selective medium. (See Clough, S. J. and Bent, A. F. (1998) Floral dip: a simplified method for Agrobacterium-mediated transformation of Arabidopsis thaliana. Plant J. 16, 735-743, and Labra, M., Vannini, C., Grassi, F., et al. (2004) Genomic stability in Arabidopsis thaliana transgenic plants obtained by floral dip. Theor. Appl. Genet. 109, 1512-1518 http://www.floraldip.com.)

### Example 2

### Transformation Oilseeds (B. napus CV Westar) Mediated by Agrobacterium tumefaciens

Seeds from B. napus CV Westar were surface-sterilized in a 50% bleach solution for 15 min with vigorous shaking. The seeds were then germinated on MS basal. After 7 days, the seedlings were collected and the hypocotyls were cut into 1-2 cm pieces. The hypocotyl sections were placed on MS basal medium with 1 mg/L of 2,4-D for 24 h to precondition the material. Hypocotyls were inoculated with an Agrobacterium for 30 min and co-cultivated on solid MS basal medium with 1 mg/L of 2, 4-D for 3 days. Plant tissue was moved to the same media containing 150 mg/L of timintin to kill the Agrobacterium, and 25 mg/L of kanamycin to select for transformed cells. After 7 days, the hypocotyls were transferred to basal medium containing 4 mg/L of 6-BAP, 0.4 mg/L of NAA, 0.5 mg/L of GA3, 5 mg/L of silver nitrate, and the above antibiotics, for organogenesis. (See Cardoza, V. and C. N. Stewart. Increased Agrobacterium-mediated transformation and rooting efficiencies in canola (Brassica napus L.) from hypocotyl segment explants. Plant Cell Rep. 2003. 21:599-604, and Priti Maheshwari, Gopalan Selvaraj, Igor Kovalchuk. Optimization of Brassica napus (canola) explant regeneration for genetic transformation, New Biotechnology, 2011, 29(1)144-156.)

### Example 3

### Transformation of Poplar

Poplar (Populus tomentosa) was also transformed, using the methods described in Cseke L J, Cseke S B, Podila G K. High efficiency poplar transformation. Plant Cell Rep, 2007, 26(9):1529-38, incorporated by reference herein in its entirety.

Materials

Materials used in the transformation included (1) young sterile poplar (Populus tomentosa) plants; and (2) Agrobacterium strains EHA105; plasmid: pCAMBIA0386-RPA123 and pCAMBIA0386-atNRT2.1.

Explants Preparation

Healthy fully expanded leaves were taken from tissue culture grown poplar plant. The leaves were then cut into 0.3-0.5 cm squares. The leaf disks were transferred into a sterile 50-ml corning tube, which contained about 2 ml liquid Co-cultivation medium (2.41 g/L WPM+30 g/L Sugar+20 mg/L Acetosyringone).

Bacterial Suspension

Agrobacterium cells were collected from liquid culture using a centrifuge at 2,500×g for 1 min. The liquid was discarded, and the bacterial pellet was suspended using the liquid Co-cultivation medium. The suspension was then diluted to an O.D.600 of 0.5-0.8.

Infection

The bacterium suspension was transferred to a corning tube with the explants. The leaf explants were incubated with bacterium at 28° C. with a gentle shaking at 100 rpm for 20 min. The bacterium suspension was then poured off. And the explants were transferred, 15-20/petri-dish, to the plates with Co-cultivation solid medium (2.41 g/L WPM+30 g/L Sugar+7 g/L Agarose+20 mg/L Acetosyringone).

Co-Cultivation

Co-cultivation was carried out at 25° C. in the dark for 3 days.

Callus Induction

The explants were transferred, 12 pieces/dish, to the callus inducing medium (2.41 g/L WPM+30 g/L Sugar+7 g/L Agarose+2 mg/L 6-BA+1 mg/L NAA+30 mg/L Kanamycin+150 mg/L Timentin). The explants were then cultured at 25° C. in the dark for 3-4 weeks.

Shoot Induction

The explants were transferred, 10 pieces/dish, to the shoot-inducing medium (2.41 g/L WPM+30 g/L Sugar+7 g/L Agarose+1 mg/L 6-BA+0.1 mg/L NAA+40 mg/L Kanamycin+Timentin 150 mg/L). The explants were then cultured at 25° C. at 16-hours photoperiod for 3-4 weeks until shoots were produced.

Root Induction

2 cm or taller shoots were cut from the explants and transferred to a root formation medium (2.41 g/L WPM+30 g/L Sugar+7 g/L Agarose+50 mg/L Kanamycin+150 mg/L Timentin). These shoots were cultured at a 16-hour photoperiod for 3-4 weeks until roots were produced and the plants reached to 12 cm in height.

Acclimation and Transplantation

Well-rooted plantlets were removed from culture medium, agarose was washed off with tap water and the plantlets were kept in the box with roots in water for 3 days. The acclimatized plants were subsequently grown in larger pots filled with soil.

### Example 4

In Example 5 (below), transgenic canola plants (and portions thereof) were exposed to deer. The canola plants were transformed by known methods, as disclosed by Priti Maheshwari, Gopalan Selvaraj, Igor Kovalchuk. Optimizations of Brassica napus (canola) explant regeneration for genetic transformation. New Biotechnology, 2011, 29(1)144-155, incorporated by reference herein in its entirety; and Cardoza, V. and C. N. Stewart. Increased Agrobacterium-mediated transformation and rooting efficiencies in canola (Brassica napus L.) from hypocotyl segment explants. Plant Cell Rep, 2003. 21:599-604, incorporated by reference herein in its entirety.

The studies described in Example 5 (below) used transgenic canola plants including a LECRPA1+2+3 construct. Canola plants were transformed by methods such as those described herein. Polymerase chain reaction (PCR) was used to confirm the insertion of the transgenes into canola plants, and real time PCR was used to determine the expression levels of the transgenes in canola.

As described above, the confirmation of insertion of the transgenes into canola plants is shown in FIG. 4. To that end, the methods described by Ahmed et al (2009) for canola genomic DNA isolation [Ahmed, I., M. Islam, W. Arshad, A. Mannan, W. Ahmad, B. Mirza (2009): High-quality plant DNA extraction for PCR: an easy approach. Journal of Applied Genetics. 50: 105-107, incorporated by reference herein in its entirety] and the method by Chen et al (2006) for PCR reactions [Chen Y., L. Lu, W. Deng, X. Yang, R. McAvoy, D. Zhao, Y. Pei, K. Luo, H. Duan, W. Smith, C. Thammina, X. Zheng, D. Ellis, Y. Li (2006): In vitro regeneration and Agrobacterium-mediated genetic transformation of Euonymus alatus Plant Cell Reports. 25(10):1043-51, incorporated by reference herein in its entirety] were used. Basically, to isolate genomic DNA from the canola plant, 2 grams of leaf tissues were used in extracting genomic DNA therefrom using an extraction buffer (100 mM Tris-HCl, 100 mM EDTA, 250 mMNaCl) and 1.5-mL microfuge tubes, followed by cell lysis with 20% SDS, and DNA extraction with phenol: chloroform: iso-amyl alcohol (25:24:1). Hydrated ether was then used to remove polysaccharides and other contaminants from the DNA preparation. Taq DNA polymerase and three sets of primer sequences were then used to amplify the three genes (LECRPA1, LECRPA2, and LECRPA3) independently via PCR.

The primer sequences used in this Example were as follows:

pRPA1-F:
[SEQ. ID. NO. 12]
ATGACTTCCTACAACTTC,
pRPA1-R:
[SEQ. ID. NO. 13]
TCATGCAGCATAGGTTGA,
(product size 858 bp);
pRPA2-F:
[SEQ. ID. NO. 14]
ATGGCTTCCTACAAGTTC,
pRPA2-R:
[SEQ. ID. NO 15]
TCATGCAGCATAGGTTGA,
(product size 861 bp); and
pRPA3-F:
[SEQ. ID. NO. 16]
ATGCTCATAAGTTTCTTTG,
pRPA3-R:
[SEQ. ID. NO. 17]
TCATGCATAGCTTGCAAT,
(product size 783 bp).

For the PCR conditions, a reaction mix was prepared in accordance with the following Table 1:

| Reagent | Volume | Final Conc. |
|---|---|---|
| 2 X CloneAmpHiFi PCR | 10 μl | 1X |
| Premix | 0.5 μl | 0.25 μM |
| Primer 1 (10 μM) | 0.5 μl | 0.25 μM |
| Primer 2 (10 μM) | 0.5 μl (<100 ng) | |
| DNA Template | 8.5 μl | |
| Sterilized distilled water | | |
| Total volume per | 20.0 μl | |
| reaction | | |

The reaction mixture(s) was then placed in a thermocycler under the following parameters: (1) 98° C. for 3 minutes; (2) 34 cycles of 98° C. for 10 seconds, 68° C. for 15 seconds, and 72° C. for 1 minute; (3) 72° C. for 5 minutes; and (4) a hold at 12° C. (the hold may last indefinitely). PCR products were then analyzed with gel electrophoresed with the Ti plasmid DNA eliminated from the template before PCR reactions were conducted [Chen et al (2006)] (products were run on an agarose gel to check the result and DNA fragments were extracted from the agarose gel). The presence of the three transgenes indicates that the canola plants produced were transgenic—these results are shown in FIG. 2.

The insertion of the transgenes into the genome of the canola plants was further verified by real time PCR—and particularly used to determine the expression levels of the inserted genes in the canola plants. To that end, total RNA of transgenic canola plants was prepared using the RNeasy Plant Mini Kit (commercially available from Qiagen, Valencia, Calif.) according to the manufacturer's instructions. RNase-free DNase set (Qiagen) was used to eliminate genomic DNA contamination of all the RNA samples. The following protocol was used:

1) Determine the amount of plant material. Do not use more than 100 mg.
2) Immediately place the weighed tissue in liquid nitrogen, and grind thoroughly with a mortar and pestle. Decant tissue powder and liquid nitrogen into an RNase-free, liquid-nitrogen-cooled, 2 ml microcentrifuge tube. Allow the liquid nitrogen to evaporate, but do not allow the tissue to thaw. Proceed immediately to step 3.
3) Add 450 μl Buffer RLT to a maximum of 100 mg tissue powder. Vortex vigorously. Short 1-3 min incubation at 56° C. may help to disrupt the tissue.
4) Transfer the lysate to a QIoAshredder spin column (lilac) placed in a 2 ml collection tube, and centrifuge for 2 min at full speed. Carefully transfer the supernatant of the flow-through to a new microcentrifuge tube without disturbing the cell-debris pellet in the collection tube. Use only this supernatant in subsequent steps.
5) Add 0.5 volume of ethanol (100%) to the cleared lysate, and mix immediately by pipetting. Do not centrifuge. Proceed immediately to step 6.
6) Transfer the sample, including any precipitate that may have formed, to an RNeasy spin column (pink) placed in a 2 ml collection tube (supplied). Close the lid gently, and centrifuge for 15 s at 8000×g (10,000 rpm). Discard the flow-through.
7) Add 700 μl Buffer RW1 to the RNeasy spin column. Close the lid gently, and centrifuge for 15 s at 8000×g (10,000 rpm) to wash the spin column membrane. Discard the flow-through.
8) Add 500 μl Buffer RPE to the RNeasy spin column. Close the lid gently, and centrifuge for 15 s at 8000×g (10,000 rpm) to wash the spin column membrane. Discard the flow-through.
9) Add 500 μl Buffer RPE to the RNeasy spin column. Close the lid gently, and centrifuge for 2 min at 8000×g (10,000 rpm) to wash the spin column membrane.
10) Optional: Place the RNeasy spin column in a new 2 ml collection tube (supplied), and discard the old collection tube with the flow-through. Close the lid gently, and centrifuge at full speed for 1 min.
11) Place the RNeasy spin column in a new 1.5 ml collection tube (supplied). Add 30-50 μl RNase-free water directly to the spin column membrane. Close the lid gently, and centrifuge for 1 min at 8000×g (10,000 rpm) to elute the RNA.

Total RNA was isolated from the leaves using the QIAGEN RNA isolation kit. And then 1 ug RNA was used to synthesize cDNAs with a cDNA synthesis kit (known to those skilled in the art). One tenth of the volume of the first-strand cDNA reaction was used for RT-PCRs to amplify the LECRPA3 gene (using the primers—RPA3-F: CACCAACTTCACCAGAGGTG [SEQ. ID. NO. 18], RPA3-R: CACGGCCTGTATTCCAAGTAGG [SEQ. ID. NO. 19]).

UBC9 (ubiquitin-conjugating enzyme 9) was used as a reference gene (see Chen X, Truksa M, Shah S, Weselake R J. A survey of quantitative real-time polymerase chain reaction internal reference genes for expression studies in Brassica napus. Anal Biochem. 2010, 405(1):138-40, incorporated by reference herein in its entirety). Primers used for the reference gene were UBC9-F:GCATCTGCCTCGACATCTTGA [SEQ. ID. NO. 20], UBC9-R:GACAGCAGCACCTTGGAAATG [SEQ. ID. NO. 21].

For real time PCR, each 20 microliter (ul) reaction mixture was as follows for production of cDNA:

| Material | Volume | |
|---|---|---|
| 5X cDNA systhesis buffer | 4 | ul |
| dNTP Mix | 2 | ul |
| RNA primer | 1 | ul |
| RT enhancer | 1 | ul |
| Verso enzyme mix | 1 | ul |
| Template (RNA) | 1-5 | ul |
| Water, nuclease-free (#R0581) | To 20 | ul |
| Total Volume | 20 | ul |

cDNA synthesis was then run at 42° C. for 30 min (for 1 cycle), and inactivation was achieved at 95° C. for 2 minutes (for 1 cycle).

20 ul reaction of real time PCR was as follows: (1) 2× EvaGreen SUPERMIX—10 ul, (2) Sterile Water—7 ul, (3) 10 uM primer 1-1 ul, (4) 10 uM primer 2-1 ul.

Final primer concentration=500 nm each. Add 1 ul template/20 ul reaction (1-10 ng cDNA)

The protocol used in the real time PCR was as follows: Real-Time PCR was performed in an optical 96-well plate with BIORAD CFX96 real time PCR machine and universal cycling conditions (98° C. for 3 min, 40 cycles of 5s at 98° C. and 60° C. for 5 s) in final volume of 20 ml.

A no template control (NTC) was also included in each run for each gene. Experiment was conducted in three technical replicates. The normalized expression of RPA3 gene was automatically determined for each reaction using the BioRAD CFX96 manager 2.0 software.

As can be seen from FIG. 3, expression was pronounced in Lines 12, 13, and 25. These lines (particularly line 13) were used in subsequent experiments (as described in greater detail below).

### Example 5

### Study Protocol for Assessing the Palatability of Genetically Modified Canola with White-Tailed Deer at the Penn State Deer Research Center

For this study, 8 adult does were used for the experimental periods. Each experimental period will consist of a “first” and “second” phase. In this Example, the “first” phase has been completed, but the “second” phase has not (i.e., the description of the second phase is that of a phophetic example).

Pre-Trial: (8 Deer, 2-4 Days) All deer were exposed to all species of plant material included in the trial for 2-4 days prior to the start of individual trials. During this initial acclimation period, (8) adult does were moved to a one acre pen where they had access to a normal diet of commercial pellets and alfalfa hay plus the plant material for the trial. (Canola—line12, Canola—line13, Canola—Wild-Type, Black Locust, and Apple). During the Pre-trial period deer were monitored twice daily during herd checks by facility personnel.

Trial: (2 Deer, 24 Hours) For each individual trial period, two does will be sorted in the handling barn and turned out to a one acre pen where they will have access to their normal diet and additional browse for the trial. Browse will consist of an equal amount of the following: Canola—line12, Canola—line 13, Canola—Wild-Type. Black Locust and Apple browse will also be included. Each individual browse sample will be photographed in front of a grid pattern both pre and post-trial. This will allow the amount of vegetation consumed during the trial to be estimated. To determine the order of plant preference, all browsing behavior will be recorded with a motion and heat activated video camera. Videos will be analyzed and all sniffing and biting events will be recorded. For each experimental period, (4) pairs of does are used, with each pair being used for 24 hours.

Results:

Based on general observations of the plants during the “first” phase, it does not appear that the deer ate much canola during the two day period. Browsing was mostly limited to apple and to some extent black locust. There are a significant number of sniffing events at the canola plants and at least one doe ate 2-3 leaves of a transgenic canola plant. However, the deer mostly ate apple as opposed to the canola.

While the various aspects of the present invention have been disclosed by reference to the details of various embodiments of the invention, it is to be understood that the disclosure is intended as an illustrative rather than in a limiting sense, as it is contemplated that modifications will readily occur to those skilled in the art, within the spirit of the invention and the scope of any claims that appear in this or any subsequent application.

What is claimed is:

1. A transgenic plant comprising within its genome at least one gene that, when expressed, provides resistance to an herbivore.

2. The transgenic plant of claim 1, wherein the at least one gene is chosen from LECRPA1, LECRPA2, and LECRPA3.

3. The transgenic plant of claim 2, wherein the at least one gene has a sequence chosen from [SEQ. ID. NO. 1], [SEQ. ID. NO. 2], [SEQ. ID. NO. 3], [SEQ. ID. NO. 4], [SEQ. ID. NO. 5], [SEQ. ID. NO. 6], and variants thereof.

4. The transgenic plant of claim 1, further comprising within its genome a gene promoter that initiates transcription of the at least one gene.

5. The transgenic plant of claim 4, wherein the gene promoter is chosen from an Alfalfa RbcS gene promoter and a 35S CaMV gene promoter.

6. The transgenic plant of claim 5, wherein the gene promoter is an Alfalfa RbcS gene promoter, and has a sequence of [SEQ. ID. NO. 7] and variants thereof.

7. The transgenic plant of claim 1, further comprising within its genome a Rubisco small subunit transit peptide.

8. The transgenic plant of claim 7, wherein the Rubisco small subunit transit peptide has a sequence chosen from [SEQ. ID. NO. 8], [SEQ. ID. NO. 9], and variants thereof.

9. The transgenic plant of claim 1, wherein the at least one gene codes for an SEA protein.

10. The transgenic plant of claim 9, wherein the at least one gene has a sequence chosen from [SEQ. ID. NO. 1], [SEQ. ID. NO. 2], and variants thereof.

11. A fruit or a seed of the transgenic plant of claim 1.

12. An expression cassette for insertion into the genome of a plant, the cassette comprising (a) two or more genes that, when expressed, provides resistance to an herbivore, and (2) at least one regulatory nucleic acid sequence.

13. The expression cassette of claim 12, wherein the two or more genes include any combination of two or more of LECRPA1, LECRPA2, and LECRPA3.

14. The expression cassette of claim 13, wherein the at least one regulatory nucleic acid sequence is a gene promoter chosen from an Alfalfa RbcS gene promoter and a 35S CaMV gene promoter.

15. The expression cassette of claim 14, further comprising a Rubisco small subunit transit peptide.

16. An expression cassette for insertion into the genome of a plant, the cassette comprising (a) at least one gene that, when expressed, provides resistance to an herbivore, and (2) at least one regulatory nucleic acid sequence.

17. The expression cassette of claim 1, wherein the at least one gene codes for an SEA protein.
